(19) United States
(12) Patent Application Publication
(10) Pub. No.: US 2011/0113859 A1
(43) Pub. Date: May 19, 2011

(54) LOW-VOLTAGE THIN-FILM FIELD-EFFECT TRANSISTORS

(75) Inventors: Thomas Anthopoulos, London (GB); Donal Donat Conor Bradley, Buckinghamshire (GB); Aneeqa Bashir, Middlesex (GB); Paul Henrich Wobkenberg, London (GB)

(73) Assignee: Imperial Innovations Limited, Lomdon (GB)

(21) Appl. No.: 13/002,703

(22) PCT Filed: Jan. 14, 2010
(86) PCT No.: PCT/GB09/01687

§ 371 Date: Jan. 26, 2011

(30) Foreign Application Priority Data
Jul. 8, 2008 (GB) 0812499.2

(51) Int. Cl. H01L 29/786; H01L 21/336; G01N 33/00

(52) U.S. Cl. 73/31.06; 438/158; 257/347; 257/E21.409; 257/E29.273

(57) ABSTRACT

A low-voltage thin-film field-effect transistor is formed by forming a gate, forming a dielectric layer on the surface of the gate, forming a source region and a drain region, and forming a semiconductor layer adjacent the dielectric layer. The dielectric layer is formed as a native oxide layer by oxidizing the surface of the gate. The semiconductor layer is deposited by spray pyrolysis. The dielectric layer may be functionalized with a self-assembling monolayer dielectric layer. The dielectric layer may be formed as a self-assembling monolayer, without first forming a native oxide (or other) dielectric layer.

This invention relates, in part, to thin-film field-effect transistors for low-voltage and low-power applications, and a fabrication method thereof.

### BACKGROUND TO THE INVENTION

Semiconducting thin-film transistors (TFTs) comprise a substrate, a semiconducting layer, a dielectric layer, and conducting materials for the source, drain and gate electrodes. Depending on the gate potential (V_{G}) and the drain potential (V_{D}), the channel current (i.e. the current flowing from the source electrode to the drain electrode, often referred to as I_{D}) can be modulated.

Advances in solution-processable TFTs are sought for the realisation of high throughput yet low-cost electronic applications. Examples include integrated logic circuits [1] and backplanes for conventional as well as novel, e.g. flexible etc, optical displays [2]. Unfortunately, the vast majority of solution-processed TFTs incorporating either organic/polymeric or inorganic semiconductors require high operating voltages, typically in the range of tens of volts. Paramount to the successful implementation of TFT technology in future device applications will be low-voltage, low-power operation. Such capabilities could enable widespread adoption of the technology in numerous low-powered portable (i.e. battery powered) applications. A number of efforts have achieved this strategic goal but typically at the cost of solution-processability of the semiconductor and/or the gate dielectric layers. The primary advantage associated with solution processing is relatively simple fabrication and potentially lower manufacturing cost.

The best examples of low-voltage TFTs reported in the literature come from devices employing self-assembled monolayer (SAM) gate dielectrics using organic as well as inorganic semiconductors [3,4]. Collet et al. [5], for example, used carboxyl terminated tetradecylenyltrichlorosilane SAMs on Si wafers for the fabrication of OFETs using evaporated α-sexithiophene. Transistor operation at 2 V was demonstrated with gate leakage currents of the order 10⁻⁶ A/cm². Other examples include pioneering work by Klauk et al. [6] where they demonstrated low-voltage organic TFTs based on octadecylphosphonic acid (ODPA) SAMs as the gate dielectric employing evaporated organic semiconductors. More recently, Ma et al. [7] reported the use of alkyl- and anthryl-alkyl-phosphonic acid SAMs also in combination with evaporated films of organic semiconductors. Unfortunately, organic TFTs are characterised by relatively low carrier mobilities that limit the range of potential applications. Recently alternative material systems have been explored including a number of metal oxide semiconductors. Ju et al. [8], for example, have demonstrated low operating voltage TFTs employing metal oxide semiconductors deposited by sputtering [9], as well as low voltage TFTs based on metal oxide nanowires that have been deposited/fabricated on a different substrate and transferred to the transistor array.

There is therefore a desire for TFTs that are (a) solution-processable in order to facilitate manufacture; whilst (b) operable at low voltages and thus suitable for low-power (e.g. portable, battery-powered etc.) applications; and (c) based on an inorganic (rather than organic or partly-organic) semiconductor layer in order to provide relatively high carrier mobilities.

### SUMMARY OF THE INVENTION

According to a first aspect of the present invention there is provided a method as defined in Claim 1 of the appended claims. Thus there is provided a method of forming a low-voltage thin-film field-effect transistor comprising: forming a gate; forming a dielectric layer on the surface of the gate; forming a source region and a drain region; and forming a semiconductor layer adjacent the dielectric layer; wherein the dielectric layer is formed as a native oxide layer by oxidising the surface of the gate; and wherein the semiconductor layer is deposited by spray pyrolysis. The order in which the various processes appear in the preceding sentence should not be interpreted as implying any particular sequence of steps in the formation of the transistor.

The native oxide dielectric layer provides multiple benefits. It is simple to form; a separate deposition step for forming the dielectric is not required, thus facilitating fabrication of the TFT. Moreover, the native oxide layer can function as an ultra-thin gate dielectric layer, thereby increasing the geometrical capacitance of the transistor channel. Since the current flowing through the semiconducting channel is directly proportional to the geometrical capacitance of the channel, this enables the operating voltage, and hence the power consumption of the transistor, to be reduced drastically. A further benefit is that the native oxide layer provides suitable surface chemistry whereby functionalisation with self-assembling monolayer (SAM) materials can be performed. Such SAM dielectric materials can provide extra insulation so that the gate leakage current is minimised.

The use of spray pyrolysis to form the semiconductor layer renders the TFT solution-processable, thereby facilitating manufacture.

Preferable, optional, features are defined in the dependent claims.

Thus, the semiconductor layer may comprise a material selected from a group comprising: oxides; oxide-based materials; mixed oxides; metallic type oxides; group I-IV, II-VI, III-VI, IV-VI, V-VI and VIII-VI binary chalcogenides; and group I-II-VI, II-II-VI, II-III-VI, II-VI-VI and V-II-VI ternary chalcogenides. Inorganic materials which fall within this group provide relatively high carrier mobilities in comparison to organic or partly-organic semiconductor layers.

Alternatively, the semiconductor layer may comprise one of the semiconductor materials disclosed in WO 2006/003584, i.e. a compound of any of the materials in the following group (i) together with any of the materials in the following group (ii), wherein group (i) comprises cadmium, zinc, lead, tin, bismuth, antimony, indium, copper and mercury; and group (ii) comprises sulphur, selenium and tellurium.

The native oxide layer may be formed using an oxygen plasma asher, by annealing, or by alternative means for oxidising a metallic surface (e.g. treating with ultraviolet light in the presence of oxygen and/or water etc.) that will be apparent to those skilled in the art.

Annealing may be performed at a temperature in the range of 100° C. to 600° C., for a time in the range of 1 second to 24 hours.

Preferably the native oxide layer is of the order of 10 nm or less in thickness. This small thickness (compared with conventional TFT dielectrics) dramatically increases the geometrical capacitance of the transistor (e.g. to around 0.4-1 μF/cm², compared with 15-20 nF/cm² for a 200 nm thick SiO₂ dielectric). This enables the magnitude of the operating voltage of the transistor to be reduced to absolute values below 2 V (i.e. values below |2| V). This is roughly 20-50 times lower than the operating voltages for conventional TFTs. As a result, the power consumption of these devices during operation is also reduced by a similar factor.

Preferably the semiconductor layer is deposited using a precursor solution. The precursor solution may be doped in order to incorporate dopant atoms in the semiconductor material once formed, which in turn may give rise to hole and/or electron transport and/or to higher charge carrier mobility in the semiconductor layer and/or induce/enhance additional physical properties. The dopant atoms may be selected from a group comprising: aluminium, indium, gallium, molybdenum, boron, nitrogen, lithium. Other dopants are also possible.

The spray pyrolysis may be performed at any suitable temperature according to the semiconductor material being deposited and the substrate used. The temperature may be in the range of 100° C. to 600° C., preferably in the range of 100° C. to 400° C., and more preferably in the range of 200° C. to 400° C.

The semiconductor layer may be sprayed in a raster fashion, although other deposition techniques are also possible.

The semiconductor layer may be deposited in a pulsed manner. By providing a time delay between successive sprays, this enables the solution vapours to adsorb to the substrate and convert to the semiconductor material.

Alternatively the semiconductor layer may be deposited in a continuous manner.

The method may further comprise heat treating the semiconductor layer to remove residual un-reacted precursor material(s).

In certain embodiments to be described below, the gate is formed of aluminium and thus the dielectric layer is formed of aluminium oxide. However, many alternative materials are possible.

Likewise, in the embodiments to be described below, the semiconductor layer is formed of zinc oxide. Again, many alternative materials are possible.

The method may further comprise functionalising the dielectric layer with a self-assembling monolayer dielectric layer. This improves the electrical insulation of the dielectric layer and, in turn, improves the operating characteristics of the TFT.

The self-assembling monolayer may be formed from constituent molecules, each of which comprises an organic chain with a functional group at one end. In the present embodiments, the self-assembling monolayer is formed from octadecylphosphonic acid, which we have demonstrated to be thermally stable and suitable for use in conjunction with spray pyrolysis for the deposition of the semiconductor layer. Other suitable self-assembling molecules may be used instead of octadecylphosphonic acid.

According to a second aspect of the present invention there is provided a method of forming a low-voltage thin-film field-effect transistor comprising: forming a gate; forming a dielectric layer adjacent the gate; forming a source region and a drain region; and forming a semiconductor layer adjacent the dielectric layer; wherein the dielectric layer is formed as a self-assembling monolayer; and wherein the semiconductor layer is deposited by spray pyrolysis. Again, the order in which the various processes appear in the preceding sentence should not be interpreted as implying any particular sequence of steps in the formation of the transistor.

By virtue of the self-assembling monolayer serving as the dielectric layer, there is no need to form an oxide (or other) dielectric layer first. This simplifies the fabrication of the TFT.

According to a third aspect of the present invention there is provided a low-voltage thin-film field-effect transistor comprising: a source region; a drain region; a semiconductor layer disposed between the source and drain regions; a gate region; and a dielectric layer disposed between the semiconductor layer and the gate region; wherein the dielectric layer is a native oxide layer formed on the surface of the gate region.

The term “between”, in the context of “the semiconductor layer disposed between the source and drain regions”, should be interpreted broadly, to encompass both spatially between (i.e. physically located between the source and the drain regions) and also functionally between (i.e. arranged to form a semiconducting channel from the source to the drain via the semiconductor layer).

According to a fourth aspect of the present invention there is provided a low-voltage thin-film field-effect transistor comprising: a source region; a drain region; a semiconductor layer disposed between the source and drain regions; a gate region; and a dielectric layer disposed between the semiconductor layer and the gate region; wherein the dielectric layer is a self-assembled monolayer.

According to a fifth aspect of the present invention there is provided an integrated circuit comprising one or more transistors in accordance with the third or fourth aspects of the invention.

According to a sixth aspect of the present invention there is provided a two-dimensional sensor array comprising a plurality of transistors in accordance with the third or fourth aspects of the invention.

According to a seventh aspect of the present invention there is provided an ultraviolet detector comprising one or more transistors in accordance with the third or fourth aspects of the invention, the said transistor(s) being situated such that ambient light (i.e. the light to be detected or measured) can reach the said transistor(s).

According to an eighth aspect of the present invention there is provided a gas sensor comprising: one or more transistors in accordance with the third or fourth aspects of the invention, wherein the transistor(s) is/are situated such that ambient gas (i.e. the gas to be detected or measured) can come into contact with the said transistor(s); means for illuminating the transistor(s) with electromagnetic radiation; and means for measuring the recovery time of the channel current of the transistor(s) following illumination by the illuminating means.

Preferably the said electromagnetic radiation is ultraviolet light.

According to a ninth aspect of the present invention there is provided a gas sensor comprising: one or more transistors situated such that an ambient gas (i.e. the gas to be detected or measured) can come into contact with the said transistor(s); means for illuminating the transistor(s) with electromagnetic radiation; and means for measuring the recovery time of the channel current of the transistor(s) following illumination by the illuminating means.

Preferably the said electromagnetic radiation is ultraviolet light, and preferably the or each transistor is a thin-film field-effect transistor.

Finally, according to a tenth aspect of the present invention there is provided an ultraviolet detector comprising one or more thin-film field-effect transistors, wherein the said transistor(s) is/are situated such that ambient light can reach the said transistor(s).

With the ninth and tenth aspects of the invention, preferably the or each transistor comprises a semiconductor layer formed of zinc oxide.

### BRIEF DESCRIPTION OF THE DRAWINGS

Embodiments of the invention will now be described, by way of example only, and with reference to the drawings in which:

FIG. 1 illustrates a schematic representation (not to scale) of a bottom-gate bottom-contact TFT architecture according to an embodiment of the invention;

FIG. 2 illustrates the experimental setup of the spray pyrolysis technique employed in this work;

FIG. 3 illustrates schematic representations of the ZnO TFT architectures employed, namely (a) a bottom-gate top-contact (BG-TC) TFT architecture, and (b) a bottom-gate bottom-contact (BG-BC) TFT architecture;

FIG. 4 illustrates alternative TFT architectures which may be used in further embodiments of the invention, namely (a) a bottom-gate top-contact TFT, (b) a top-gate top-contact TFT, (c) a top-gate bottom-contact TFT, (d) a top-gate TFT with asymmetric source-drain contacts, and (e) a double gate TFT;

FIG. 5 illustrates the chemical structure of zinc acetate di-hydrate precursor material;

FIG. 6 illustrates experimentally-measured transfer curves for a ZnO TFT (channel length L=60 μm, channel width W=2 mm) based on a native aluminium oxide (AlO_{X}) gate dielectric, with the ZnO having been deposited by spray pyrolysis in ambient atmosphere at a substrate temperature of 400° C.;

FIG. 7 illustrates the molecular structure of the octadecylphosphonic acid used for the functionalisation of native aluminium oxide formed on the surface of an aluminium gate electrode;

FIG. 8 shows water droplets on (a) Al—AlO_{X} and (b) Al—AlO_{X}—ODPA surfaces;

FIG. 9 shows water droplets on (a) an as-prepared Al—AlO_{X}—ODPA surface exhibiting a contact angle (θ) of >105°, and (b) an Al—AlO_{X}—ODPA surface after thermal annealing of the substrate at 400° C. for 10 minutes, also exhibiting a contact angle of >105′;

FIG. 10 illustrates experimentally-measured (a) transfer characteristics and (b) output characteristics, in both cases for a ZnO TFT (L=60 μm, W=2 mm) based on an AlO_{X} gate dielectric, with the ZnO having been deposited by spray pyrolysis in ambient atmosphere at a substrate temperature of 400° C.;

FIG. 11 illustrates circuitry of a unipolar voltage inverter fabricated using two low-voltage ZnO TFTs where the ZnO was deposited via spray pyrolysis;

FIG. 12 illustrates examples of alternative unipolar circuits that could be used together with low-voltage oxide TFTs to realise voltage inverters;

FIG. 13 illustrates experimentally-measured transfer curves for a unipolar inverter (circuitry shown in FIG. 11) employing two low-voltage TFTs based on ZnO deposited by spray pyrolysis at a substrate temperature of 400° C.;

FIG. 14 illustrates experimentally-measured transfer characteristics of a low-voltage ZnO TFT (L=60 μm, W=1 mm) before and after illumination with a UV-LED; and

FIG. 15 illustrates an experimental evaluation of the photoenhanced drain current (I_{D}) of a ZnO TFT, after brief (10 seconds) illumination with UV light (λₚₑₐₖ=400 nm).

In the figures, like elements are indicated by like reference numerals throughout.

### DETAILED DESCRIPTION OF PREFERRED EMBODIMENTS

The present embodiments represent the best ways known to the applicants of putting the invention into practice. However, they are not the only ways in which this can be achieved.

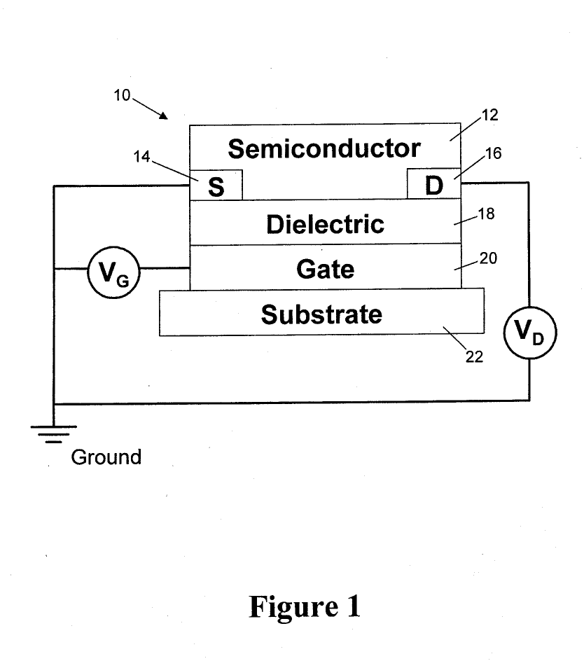

By way of introduction, as illustrated in FIG. 1, a conventional bottom-gate bottom-contact TFT device 10 consists of a semiconducting active layer 12 applied onto a three-terminal electrode architecture comprising a source electrode 14, a drain electrode 16 and a gate electrode 20. The gate electrode 20 is separated from the semiconductor layer 12 and the source and drain electrodes by a dielectric layer 18.

In embodiments of the present invention, the semiconducting layer 12 is deposited using spray pyrolysis (which may be abbreviated to “SP” herein). This builds upon our earlier work on the spray pyrolysis of semiconducting layers, as described in WO 2008/129238 (for a TiO₂ semiconducting layer) and, more recently, PCT/GB2009/001635 (which claims priority from GB 0811962.0) for other semiconducting materials, in particular ZnO. However, in the presently-preferred embodiments, the dielectric 18 is formed as a native oxide layer by oxidising the surface of the gate 20, and/or as a self-assembling monolayer, prior to the deposition of the semiconducting layer 12. Other fabrication sequences are, in principle, possible, though. For example, the semiconducting layer may be formed before the gate or dielectric.

As a proof of concept of embodiments of the present invention, we demonstrate the fabrication of low-voltage TFTs and inverter circuits based on zinc oxide (ZnO) films deposited by spray pyrolysis. Spray pyrolysis is a simple low-cost deposition technique suitable for large area deposition and could potentially enable the fabrication of large area electronics at low cost. Further details of the SP process may be obtained from PCT/GB2009/001635 (which claims priority from GB 0811962.0). Importantly, ZnO can be deposited in ambient conditions without any special precautions. Freshly prepared devices exhibit air stability and are capable of operating even after exposure to atmospheric air for several months.

### Experimental Procedures

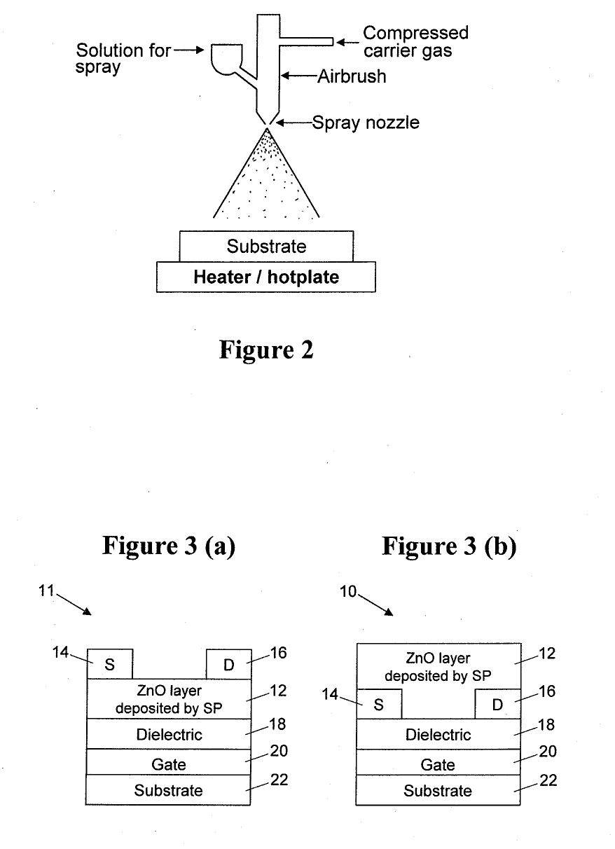

The spray pyrolysis technique may be used for the deposition of simple oxide films, mixed oxide films, metallic type oxides, group I-IV, II-VI, III-VI, IV-VI, V-VI and VIII-VI binary chalcogenides, and group I-II-VI, II-II-VI, II-III-VI, II-VI-VI and V-II-VI ternary chalcogenides. Films of such metal oxide and metallic spinel oxide materials prepared by SP have matching properties for a wide range of technological applications. Surprisingly, however, use of SP for the fabrication of TFTs is very limited. Recently, in PCT/GB2009/001635 (which claims priority from GB 0811962.0), we have demonstrated the fabrication of functional TFTs based on ZnO where the semiconductor was deposited by SP. The experimental setup of the spray pyrolysis technique used here is shown in FIG. 2.

TFTs were fabricated in different device configurations similar to the ones shown in FIGS. 3(a) and (b). For the substrate 22, a number of different materials have been studied, including plastic (i.e. flexible), glass, and silicon oxide substrates. In principle any substrate material with the required physical properties could be employed. For the experimental work described here the TFTs were fabricated on glass substrates, but different type of substrates could be used, including both inorganic as well as organic (e.g. plastics) materials. Using conventional photolithography or simple shadow masking, gate 20, source 14 (“S”) and drain 16 (“D”) electrodes were defined in a bottom-gate bottom-contact (BG-BC) configuration (FIG. 3(b)) or a bottom-gate top-contact configuration (FIG. 3(a)).

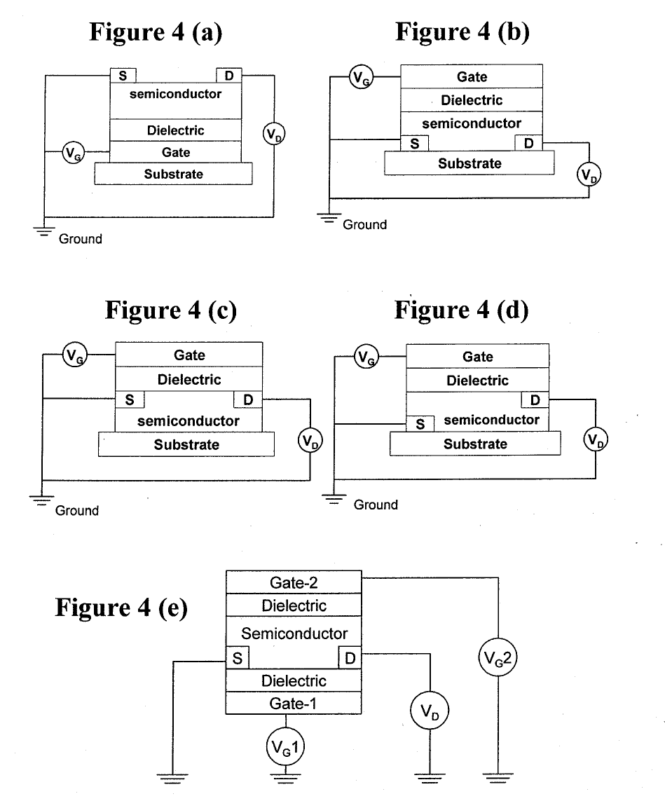

In principle many alternative transistor architectures could be employed, other than the bottom-gate bottom-contact architecture of FIGS. 1 and 3(b), or the bottom-gate top-contact architecture of FIG. 3(a). Some examples are shown in FIG. 4, which illustrates (a) a bottom-gate top-contact TFT, (b) a top-gate top-contact TFT, (c) a top-gate bottom-contact TFT, (d) a top-gate TFT with asymmetric source-drain contacts, and (e) a double gate TFT. These different device architectures, in combination with different metal electrodes, may be exploited for performance enhancement of the TFT depending on the particular application. Other device architectures are also envisioned and will be familiar to those skilled in the art.

For this work, low-voltage TFTs were fabricated employing aluminium (Al) gate electrodes that were deposited using thermal vacuum sublimation to a thickness of 5 nm to 100 μm. To form a dielectric layer, the aluminium gate electrodes were then oxidised employing a plasma asher apparatus or by simple thermal annealing in ambient air or controlled oxygen atmosphere. The duration of the plasma ashing and thermal annealing varies depending on the experimental conditions. Typical treatment times were between 1 second and 24 hours. As a consequence of the plasma/thermal treatment, a native aluminium oxide (AlO_{X}) layer was formed in the surface of the Al gate electrode. As will be explained shortly, the native AlO_{X} layer plays a dual role, as well as being simple to form. Its first role is that it can function as an ultra-thin gate dielectric layer, while its second role is to provide suitable surface chemistry where functionalisation with self-assembling monolayer materials can be performed.

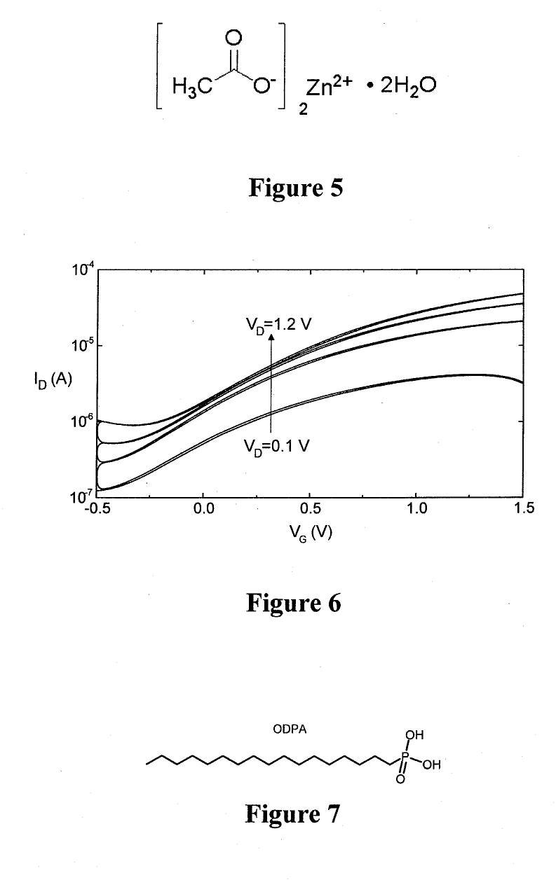

Deposition of the semiconductor was then performed. Specifically, deposition of a ZnO layer was performed using a 0.2 M methanolic precursor solution of zinc acetate di-hydrate (FIG. 5) as the channel layer, onto the gate electrodes by spray pyrolysis in ambient air, at various temperatures: 100° C., 200° C., 300° C., 400° C. and 500° C.

The film was grown in the following manner: The substrate was placed on the hot plate until the required substrate temperature was stabilised. The precursor solution was then sprayed with the airbrush positioned vertically above the substrate (FIG. 2) in a raster fashion. A time delay of 0.1-600 seconds was given before the second spray cycle (in a similar manner) to allow the solution vapours to adsorb to the substrate and convert to ZnO (or any other oxide/semiconductor material depending on the spraying solution). The process can be repeated several times until a semiconducting layer is formed.

### Semiconductor Film Materials, for Deposition by Spray Pyrolysis

In spray pyrolysis a precursor solution is dispersed and transported by means of an inert carrier gas so it arrives at the surface of the targeted substrate in the form of very fine droplets. Upon arrival the material droplets react chemically and convert to the desired chemical compound in the form of a film layer. The chemical reactants are selected such that the by-products (i.e. products formed in addition to the desired compound) are volatile at the specific deposition temperature so they do not stay on the surface of the substrate.

To the best of our knowledge, to date there are no reported examples of low-voltage oxide TFTs fabricated by SP, the most relevant work being our own patent applications WO 2008/129238, on TiO₂-based TFTs, and PCT/GB2009/001635 (which claims priority from GB 0811962.0), on TFTs based on ZnO and other materials. There are many material systems that could be suitable for the fabrication of low-voltage TFTs by spray pyrolysis. These include all precursor complexes containing cadmium (Cd), zinc (Zn), lead (Pb), tin (Sn), bismuth (Bi), antimony (Sb), indium (In), copper (Cu), mercury (Hg), sulphur (S), selenium (Se) and tellurium (Te).

Other materials that could possibly be used as inorganic semiconductors deposited by spray pyrolysis include:
1. Titanium dioxide —TiO₂ (see WO 2008/129238)
2. Magnesium oxide —MgO
3. Nickel oxide —NiO
4. Molybdenum trioxide —MoO₃
5. Tungsten oxide —WO₃
6. Iron oxide —FeO
7. Zirconium dioxide —ZrO₂
8. Or more general AMO₂ materials where A=Silver (Ag), and M=Al, Ga
9. Materials disclosed in WO 2006/003584, i.e. a compound of any of the materials in the following group (i) together with any of the materials in the following group (ii), wherein group (i) comprises cadmium, zinc, lead, tin, bismuth, antimony, indium, copper and mercury; and group (ii) comprises sulphur, selenium and tellurium.

We have proven the two important concepts of the present disclosure, namely (a) the formation of a native oxide dielectric layer, and (b) the functionalisation of the gate electrode with a self-assembled monolayer, to work with a semiconductor layer of ZnO deposited by spray pyrolysis. Our most important findings are as follows:

### Al—AlO_{X}—ZnO Transistors—to Demonstrate the Formation of a Native Oxide Dielectric Layer

The first type of device that has been fabricated and studied is based on the BG-TC architecture shown in FIG. 3(a). As the substrate material we employed either Si/SiO₂ or glass. Aluminium was employed as the gate electrode material, but other materials such as different metals as well as conductive metal oxides with suitable chemical properties could, in principle, be used. As the dielectric layer aluminium oxide was employed. Aluminium oxidation was performed either using an oxygen plasma asher or by annealing on a hot plate (annealing temperature 100-600° C. in ambient air or in the presence of water vapour). The semiconductor precursor solution was then deposited by SP with the substrate maintained at temperatures in the range 100-600° C.

FIG. 6 shows the transfer characteristics of a ZnO TFT employing an aluminium gate, a native oxide layer of AlO_{X} as the gate dielectric, ZnO deposited by SP as the semiconductor, and aluminium source and drain electrodes. As can be seen, clear transistor characteristics can be observed with the device exhibiting strong electron transporting (n-channel) behaviour. A current on/off ratio of 10-20 and a maximum electron mobility of ˜5 cm²/Vs is obtained. The gate current (I_{G}) for all measurements was less than I_{D} by more than one order of magnitude.

Surprisingly, the native aluminium oxide in combination with the spray pyrolysis technique yields functional transistors even when the as-deposited (i.e. non oxidised) aluminium gate electrodes are annealed only for a short period of time (e.g. 1 second to 5 minutes in the presence of ambient air or water vapour). The surprisingly easy fabrication of the low-voltage ZnO TFTs could be possibly traced to the aqueous nature of the precursor solution that provides, simultaneously, the Al gate electrode with the required oxygen for the formation of a native insulating layer of AlO_{X} (−1-5 nm thick), and also the required oxygen supply for the conversion of the precursor zinc acetate to ZnO. To the best of our knowledge this is the first example of spray pyrolysis combined with a solution processed metal (Zn in this specific example) acetate and ultra-thin native metal oxide (AlO_{X} in this specific example) for the fabrication of low-voltage TFTs. Surprisingly, performance of the transistors shown in FIG. 6 is among the best reported in the literature for ZnO TFTs based on a solution-processed semiconductor layer.

Al—AlO_{X}—SAM-ZnO Transistors—to Demonstrate the Functionalisation of the Gate Electrode with a Self-Assembled Monolayer

Although the Al—AlO_{X}—ZnO based transistors shown in FIG. 6 exhibit high electron mobility, the on/off ratio is relatively low while the gate leakage current (I_{G}, not shown here) is also relatively high and for V_{G}>1.5 V becomes comparable with I_{D} (approximately ten times smaller). In order to improve the transistor characteristics we have functionalised the native aluminium oxide (AlO_{X}) with a self-assembled monolayer (SAM) dielectric. The specific molecule employed is the octadecylphosphonic acid (ODPA) shown in FIG. 7. ODPA consists of a C-18 alkyl chain and a phosphoric acid group at one end. From the literature [6] it is known that such a SAM layer is capable of providing the necessary extra current insulation required.

Functionalisation of the ODPA layer was performed as follows: The aluminium gate electrodes were oxidised using an oxygen plasma asher or by annealing in atmospheric air using a hot plate (annealing temperature 100-600° C. in ambient air or in the presence of water vapour), thereby forming an aluminium oxide layer. The samples were then sonicated in ethanol and blown dry with nitrogen gas. The substrates were then immediately submerged in a beaker containing a 1-5 mM solution of ODPA in ethanol (other solvents may alternatively be used) and left to functionalise for several hours (although other periods of time, in the range of 1 second to 24 hours, are also possible). The modified substrates were sonicated in ethanol, dried in nitrogen gas and then placed in an oven at 140° C. for a period of time.

Alternatively, the SAM layer may be functionalised via simple drop casting of the SAM solution on the Al—AlO_{X} surface, followed by annealing at 140° C. and then washing in ethanol, drying in nitrogen gas, and placing in an oven at 140° C. In this way we avoid long time immersion of the substrate to the SAM solution and hence the entire process is very fast. Zschieschang et al. [10] have also demonstrated SAM functionalisation employing micro-contact printing of a SAM solution to an Al—AlO_{X} surface, and this technique may be employed in conjunction with the embodiments of the present invention.

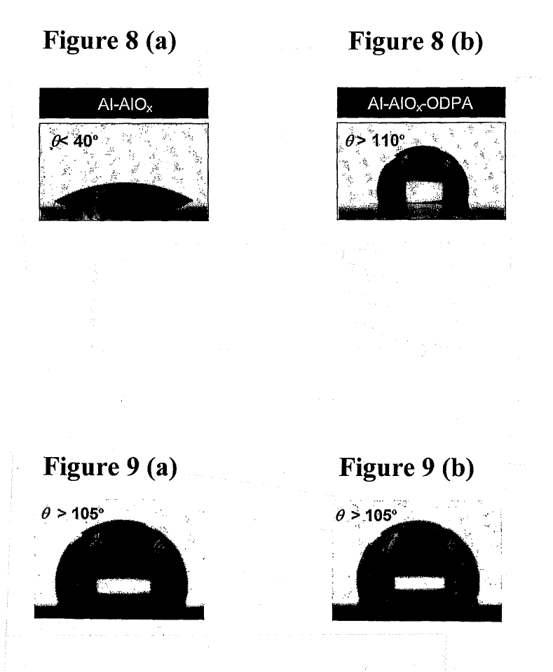

In our present work, functionalisation of the gate electrode surfaces was confirmed using liquid contact angle measurements and current-voltage measurements. FIG. 8 shows a water drop on the AlO_{X} surface before (FIG. 8(a)) and after (FIG. 8(b)) functionalisation with ODPA. A clear increase in the water drop's contact angle upon treatment of the surface is observed, confirming the presence of the ODPA SAM. It should be noted, however, that different SAM deposition procedures could be employed for the functionalisation of the ODPA, or other SAM dielectrics known from the literature (e.g. [10]), on the surface of the AlO_{X} (or other oxide surfaces). Such procedures and materials will be known or familiar to those skilled in the art.

Another important characteristic of the SAM dielectrics employed (ODPA in this particular case), and very relevant to the present work, is their thermally stable nature. FIG. 9 shows the contact angle measurements for a water drop on a freshly prepared Al—AlO_{X}—ODPA dielectric immediately after functionalisation (FIG. 9(a)) and after substrate annealing at 400° C. for 10 minutes in nitrogen (FIG. 9(b)). Clearly the ODPA monolayer is still present (as evident by the high contact angle of the water drop), even after annealing of the substrates at high temperatures for several minutes. The combination of the high insulating properties of ODPA, and possibly many other SAM materials known from the literature, and their thermal stability, make self-assembled monolayer gate dielectrics superb candidate materials for use in combination with spray pyrolysis for the fabrication of low-voltage oxide TFTs.

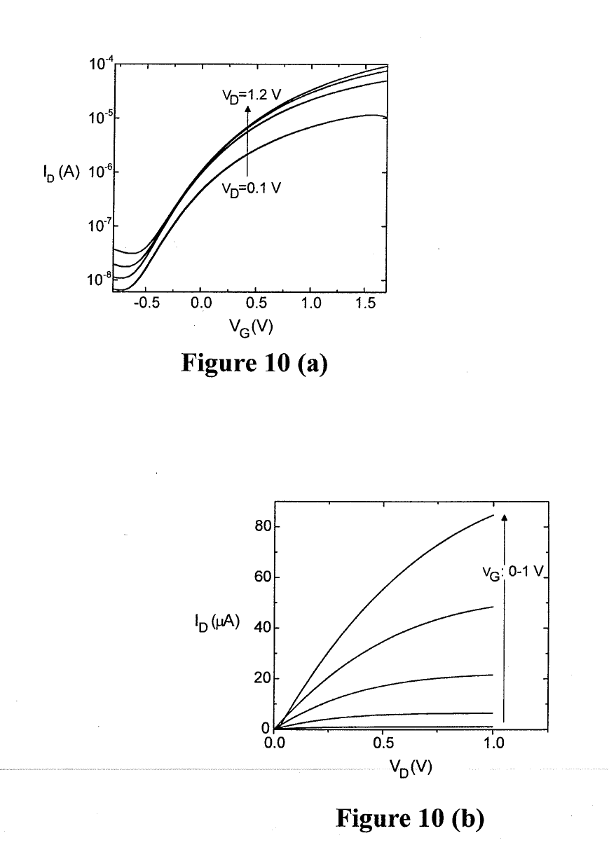

To prove that ODPA, and generally SAM dielectrics, could be used in combination with spray pyrolysis, and that can lead to improved device performance, we fabricated TFTs using Al—AlO_{X}—ODPA dielectrics and ZnO layers deposited by spray pyrolysis at substrate temperatures in the range 100-450° C. The transfer and output transistor characteristics obtained for a AlO_{X}—ODPA-based TFT are shown in FIGS. 10(a) and (b) respectively. Excellent transistor operation is observed with maximum electron mobilities in the range of 10-30 cm²/Vs, and threshold voltages of the order of V_{TH}˜0 V.

Although the functionalisation of the dielectric layer with a self-assembled monolayer is a useful technique that may be employed in conjunction with a native oxide dielectric layer, a self-assembling monolayer may be used to form a dielectric layer by itself, without needing to first form a native oxide (or other) dielectric layer. For instance, one could envision the use of conductive oxide gate electrodes, such as tin dioxide (SnO₂), doped-SnO₂ or indium tin oxide (ITO) to name a few, where the SAM dielectric is functionalised directly onto the gate electrode followed by the deposition of the oxide semiconductor layer by SP. In principle any conductive materials (metallic, oxide, or organic) with suitable surface chemistry could be employed in combination with SAM dielectrics.

### Low-Voltage Integrated Circuits Based on Oxide TFTs Fabricated Using Spray Pyrolysis

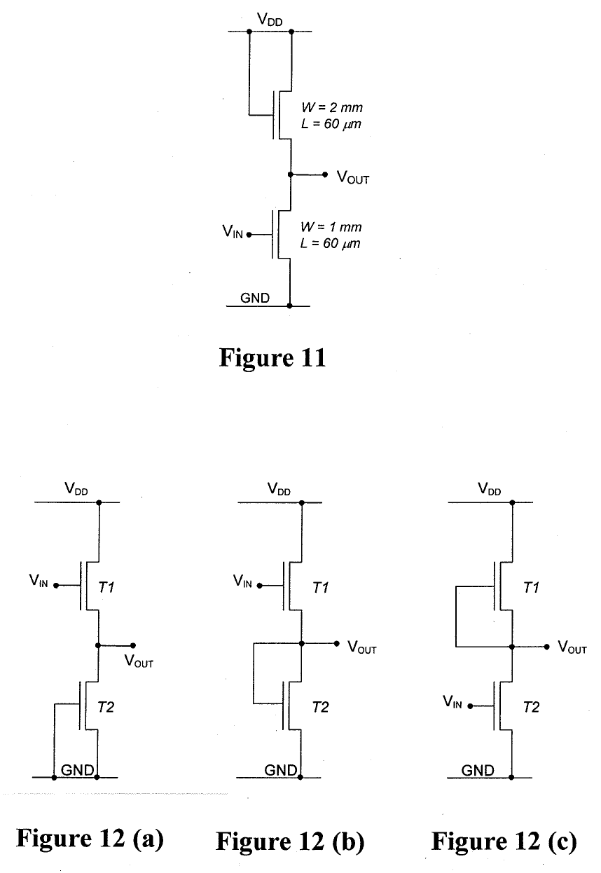

To demonstrate the potential of the low-voltage ZnO TFTs fabricated by SP in real logic circuits, we have fabricated unipolar voltage inverters. To achieve this, two BG-TC low-voltage ZnO TFTs, employing AlO_{X}—ODPA as the gate dielectric, are integrated to create the circuit shown in FIG. 11. The dimensions of the two transistors used here are not limited to those given in FIG. 11, but could be designed to any specific size required. Similarly, the specific unipolar circuitry chosen here is not limited to the one shown in FIG. 11, but could be adopted to the operating characteristics of the individual transistors used. Typical examples of other unipolar inverter circuits are shown in FIG. 12. Similarly, other transistor architectures (see for example FIG. 4) such as BG-BC, top-gate or dual-gate configurations could be employed. Such design procedures are standard and will be known to those skilled in the art.

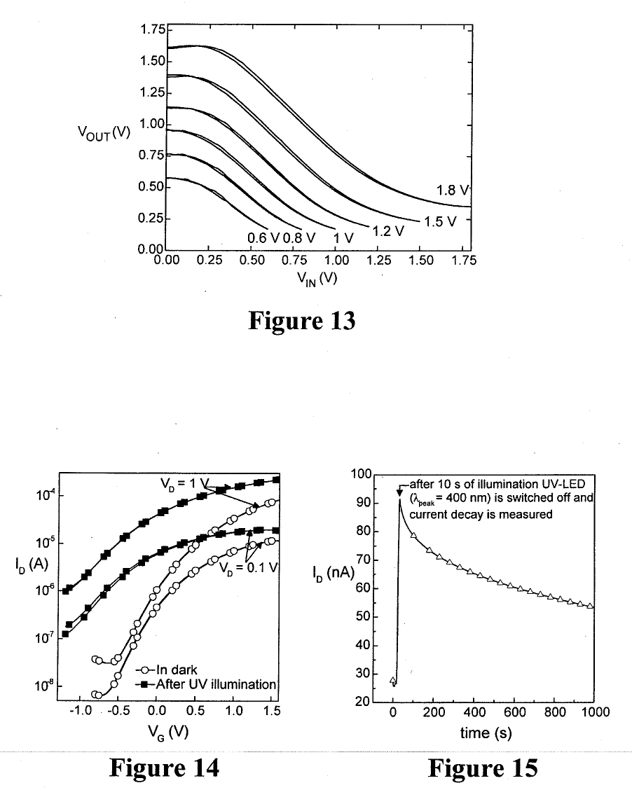

FIG. 13 shows the experimentally-measured transfer characteristics of a unipolar voltage inverter comprised of two n-channel low-voltage TFTs based on ZnO deposited by spray pyrolysis. The physical dimensions of the individual transistors are shown in FIG. 11. Excellent operating characteristics are observed, with the circuits operating with a supply voltage of less than 1.8 V. This demonstrates that our transistors are capable of low-voltage low-power operation.

### Ultraviolet Light Sensing ZnO Transistors Fabricated by Spray Pyrolysis

In addition to their electronic function, we have also established that ZnO TFTs (including high voltage as well as low-voltage TFTs) can be used as ultraviolet (UV) light detectors. Use of low-voltage ZnO TFTs for UV light detection, in particular, could be very attractive since the devices are operating at low voltages and hence the power consumed is also expected to be very low. An example of the effect of illuminating a low-voltage ZnO TFT with UV light (400 nm) is shown in FIG. 14. The transistor structure used for this particular experiment is the one shown in FIG. 3(a), but other device architectures could also be used. The LTV illumination source used for this experiments was a UV light-emitting diode (UV-LED) with a peak emission (λ) at 400 nm.

The important effect observed upon illumination of the ZnO TFTs with 400 nm light is a shift on the transfer curves toward higher drain currents (I_{D}) and more positive gate voltages (V_{G}). The shift depends both on the power density (W/cm²) of the illumination used, its wavelength (λ) and the exposure time of the TFT to the UV light. We find that when the device is illuminated with green or red light no shift in the operating curves of the transistors is observed. Photoresponse is observed only when the device is illuminated with photon energies higher than the band gap of the ZnO (for example using light wavelengths shorter than 440 nm); then a shift in the operating characteristics of the ZnO TFT is observed. In general, the shorter the light wavelength, the higher the photoresponse of the ZnO TFT observed. We have observed the same UV light induced effects with our “normal” (i.e. not low-voltage) ZnO TFTs based on thick gate dielectrics (for example SiO₂, or fluoropolymers such as CYTOP).

Another interesting finding is that when the ZnO TFTs are illuminated while maintained in nitrogen ambient, or under vacuum, the photoinduced shift in the operating curves of the transistor upon UV illumination is characterised by relatively long retention times. This means that the operating characteristics following LTV illumination do not recover (i.e. back to the operating characteristics measured before UV illumination) for a long period of time that typically exceeds 1 week. When the same test is performed under ambient air, the photoresponse is faster with recovery times ranging between 30 minutes to only few minutes. The effect is shown in FIG. 15. The recovery time when performed under ambient air depends on the initial illumination intensity of the UV light, its wavelength, exposure time and the presence of oxygen and relative humidity.

ZnO TFTs could therefore be used for the fabrication of discrete UV light sensors, and also two dimensional sensor arrays. One can also envision integration of such UV sensing ZnO TFTs with auxiliary driving transistors, similar but not limited to those shown in FIG. 12, for the fabrication of high gain UV light sensors.

By taking advantage of the dependence of the ZnO TFT photoresponse on the ambient conditions, one can also envision fabrication of gas sensors by monitoring the recovery time of the channel current (I_{D}) following illumination of the device with suitable electromagnetic radiation (e.g. ultraviolet light).

### REFERENCES

[1] Crone et al., Nature 403, 521 (2000)
[2] Gelinck et al., Nat. Materials 3, 110 (2004)
[3] Halik et al., Nature 431, 963 (2004)
[4] Yoon et al., Proc. Nat. Acad. Sci. 102, 4678 (2002)
[5] Collet et al., Appl. Phys. Lett. 76, 1941 (2000)
[6] Klauk et al., Nature 445, 745 (2007)
[7] Ma et al., Appl. Phys. Lett. 92, 113303 (2008)
[8] Ju et al., Nano Lett. 4, 997 (2008)
[9] Fortunato et al., Appl. Phys. Lett, 85, 2541 (2004)
[10] Zschieschang et al., Langmuir 24, 1665 (2008)

1-54. (canceled)

55. A method of forming a low-voltage thin-film field-effect transistor, the method comprising the steps of:
forming a gate having a surface;
forming a dielectric layer on the surface of the gate;
forming a source region and a drain region; and
forming a semiconductor layer adjacent the dielectric layer;
wherein the dielectric layer is formed as a native oxide layer by oxidizing the surface of the gate; and
wherein the semiconductor layer is deposited by spray pyrolysis.

56. The method as claimed in claim 55, wherein the semiconductor layer comprises a material selected from a group comprising:
oxides; oxide-based materials; mixed oxides; metallic type oxides; group I-IV, II-VI, III-VI, IV-VI, V-VI and VIII-VI binary chalcogenides; and group I-II-VI, II-II-VI, II-III-VI, II-VI-VI and V-II-VI ternary chalcogenides.

57. The method as claimed in claim 55, wherein the semiconductor layer comprises a compound of any of the materials in the following group (i) together with any of the materials in the following group (ii), wherein
group (i) comprises cadmium, zinc, lead, tin, bismuth, antimony, indium, copper and mercury; and
group (ii) comprises sulphur, selenium and tellurium.

58. The method as claimed in claim 55, wherein the native oxide layer is of the order of 10 nm or less in thickness.

59. The method as claimed in claim 55, wherein the semiconductor layer is deposited using a precursor solution, the precursor solution being doped in order to incorporate dopant atoms in the semiconductor material once formed.

60. The method as claimed in claim 59, wherein the dopant atoms are selected from a group comprising: aluminum, indium, gallium, molybdenum, boron, nitrogen, lithium.

61. The method as claimed in claim 55, wherein the spray pyrolysis is performed at a temperature in the range of 100° C. to 400° C.

62. The method as claimed in claim 55, wherein the gate is formed of aluminum and thus the dielectric layer is formed of aluminum oxide.

63. The method as claimed in claim 55, wherein the semiconductor layer is formed of zinc oxide.

64. The method as claimed in claim 55, further comprising functionalizing the dielectric layer with a self-assembling monolayer dielectric layer.

65. The method as claimed in claim 64, wherein the self-assembling monolayer is formed from constituent molecules, each of which comprises an organic chain with a functional group at one end.

66. The method as claimed in claim 65, wherein the self-assembling monolayer is formed from octadecylphosphonic acid.

67. A method of forming a low-voltage thin-film field-effect transistor, the method comprising the steps of:
forming a gate;
forming a dielectric layer adjacent the gate;
forming a source region and a drain region; and
forming a semiconductor layer adjacent the dielectric layer;
wherein the dielectric layer is formed as a self-assembling monolayer; and
wherein the semiconductor layer is deposited by spray pyrolysis.

68. The method as claimed in claim 67, wherein the self-assembling monolayer is formed from constituent molecules, each of which comprises an organic chain with a functional group at one end.

69. The method as claimed in claim 68, wherein the self-assembling monolayer is formed from octadecylphosphonic acid.

70. A low-voltage thin-film field-effect transistor, comprising:
a source region;
a drain region;
a semiconductor layer disposed between the source and drain regions;
a gate region having a surface; and
a dielectric layer disposed between the semiconductor layer and the gate region;
wherein the dielectric layer is a native oxide layer formed on the surface of the gate region.

71. A low-voltage thin-film field-effect transistor, comprising:
a source region;
a drain region;
a semiconductor layer disposed between the source and drain regions;
a gate region; and
a dielectric layer disposed between the semiconductor layer and the gate region;
wherein the dielectric layer is a self-assembled monolayer.

72. A gas sensor, comprising:
at least one low-voltage thin-film field-effect transistor including a source region, a drain region, a semiconductor layer disposed between the source and drain regions, a gate region, and a dielectric layer disposed between the semiconductor layer and the gate region, the dielectric layer being a self-assembled monolayer;
wherein the at least one transistor is situated such that ambient gas can come into contact with the at least one transistor;
means for illuminating the at least one transistor with electromagnetic radiation; and
means for measuring recovery time of channel current of the at least one transistor following illumination by the illuminating means.

73. The gas sensor as claimed in claim 72, wherein the electromagnetic radiation is ultraviolet light.

74. A gas sensor, comprising:
at least one transistor situated such that an ambient gas can come into contact with the at least one transistor;
means for illuminating the at least one transistor with electromagnetic radiation; and
means for measuring recovery time of channel current of the at least one transistor following illumination by the illuminating means.
